(19) United States
(12) Patent Application Publication
(10) Pub. No.: US 2016/0122262 A1
(43) Pub. Date: May 5, 2016

(54) Xylene Isomerization Process with Sulfidation

(71) Applicant: ExxonMobil Chemical Patents Inc., Baytown, TX (US)

(72) Inventors: Chunshe Cao, Conroe, TX (US); Rachel M. Cook, Houston, TX (US); Michel Molinier, Houston, TX (US); Robert G. Tinger, Friendswood, TX (US); Marcus L. van Nuland, Rijen (NL)

(21) Appl. No.: 14/861,783

(22) Filed: Sep. 22, 2015

Related U.S. Application Data
(60) Provisional application No. 62/073,625, filed on Oct. 31, 2014.

(51) Int. Cl. C07C 5/27

(52) U.S. Cl. CPC C07C 5/2732; C07C 5/277

(57) ABSTRACT

A process is provided for the isomerization of aromatic hydrocarbons including contacting an aromatic hydrocarbon feedstream with a catalyst comprising a hydrogenation metal wherein sulfur is continuously introduced after catalyst start-up for an extended period of time at a concentration of no more than 56 ppm by weight of the aromatic hydrocarbon feedstream.

### CROSS REFERENCE TO RELATED APPLICATIONS

This application claims priority to and the benefit of U.S. Provisional Application No. 62/073,625, filed Oct. 31, 2014 which is incorporated herein by reference in its entirety. This application is related to concurrently filed U.S. patent application Ser. No. ______ (U.S. Provisional Application No. 62/094117, bearing Attorney Docket No. 2014EM381/2).

### FIELD OF THE INVENTION

The invention relates to a process for producing xylenes and particularly to xylene isomerization.

### BACKGROUND OF THE INVENTION

Paraxylene (also “p-xylene” or “PX”) is generally considered the most important of C8 aromatic isomers, being used as an intermediate or starting material for such diverse end uses as synthetic fibers and bottle plastic. Processes for producing xylenes include transalkylation, disproportionation, toluene alkylation with methanol, among others, certain of which are capable of producing paraxylene selectively, i.e., in amounts greater than thermodynamic equilibrium (23 mol % based on the xylene isomers at typical processing conditions). Paraxylene, however, is typically obtained from a C8 aromatic hydrocarbon mixture derived from reformate by processes including aromatic extraction and fractional distillation. Although the composition of this starting C8 aromatic hydrocarbon mixture varies over a wide range, the mixture generally comprises 5 to 40 wt % ethylbenzene, with the balance as xylenes being divided between approximately 50 wt % meta-xylene and 25 wt % each of paraxylene and ortho-xylene (this distribution considered the nominal “equilibrium concentration” of xylenes). Paraxylene may be separated from this composition in the well-known xylenes loop, which generally involves separation from the composition typically by adsorption or crystallization, leaving behind a paraxylene-depleted stream (“raffinate”), which is isomerized by liquid or vapor phase isomerization, or a combination thereof, to an equilibrium mixture of xylenes, followed by recycle to the paraxylene separation step. Since, by some accounts, 80% or more of the end use of xylenes involves the conversion of paraxylene for the above mentioned applications, obtaining para-xylene from its C8 isomers meta-xylene, ortho-xylene, and ethylbenzene, is the subject of continuing research.

Aromatic C8 isomerization can be accomplished using catalysts with hydrogenation metals. Fresh catalysts tend to exhibit very high metal activity which can lead to elevated temperatures, high hydrogenation and hydrocracking rates if not properly managed. Conventional approaches to tempering the hyper-activity of the hydrogenation metal on hydroprocessing units include pre-sulfiding the catalysts prior to introduction of hydrocarbons (“oil-in” or “catalyst start-up”) and co-sulfiding the catalysts during oil-in and for a period of minutes or hours immediately thereafter. With either approach, the object is to decrease the level of activity of the metal sites of the catalyst. Sulfiding is conventionally limited to pre-sulfiding or co-sulfiding during and immediately after catalyst start-up for only as long as the exothermic heat of reaction is unmanageable without sulfur, because there are several conventional concerns associated with the presence of sulfur in the feed to a reaction process. First, sulfur in the feed is thought to generally result in sulfur species in the products, which may affect the quality of the product or render product storage or sale more difficult. Second, sulfur has been known to cause permanent deactivation of some catalyst sites, which may result in lower yields, higher aging rates, and shorter catalyst cycles. By way of example of limited use of sulfiding in a non-isomerization application, U.S. Pat. No. 8,242,322 claims in part “introducing a sulfur source to the transalkylation zone after 2 days on stream of a process cycle of the process . . . wherein the sulfur source is introduced intermittently”.

On occasion, it may be necessary to conduct isomerization under sub-optimal reaction conditions due to temporary or permanent system constraints. Some conditions, such as reduced hydrocarbon feed rate and/or elevated hydrogen levels, may result in excessively high reaction rate and/or reaction time. Excessive reaction rate and/or reaction residence time may lead to detrimental yield losses such as ring saturation (i.e., hydrogenation of the aromatic rings of the hydrocarbon feed) and subsequent cycloalkane hydrocracking. Conventionally, when necessary to operate under sub-optimal high-activity conditions, measures such as partial catalyst unloading or temporary unit shutdown are taken in order to limit yield losses. These measures have been taken in favor of co-sulfiding due to the conventional concerns regarding sulfiding discussed above.

### SUMMARY OF THE INVENTION

It has surprisingly been discovered that having sulfur present in an isomerization reaction zone for an extended period of time can be practiced under certain conditions, including sub-optimal highly reactive non-start-up conditions, without affecting long-term catalyst performance. This discovery is embodied in the present inventive continuous process for the isomerization of aromatic hydrocarbons which comprises contacting an aromatic hydrocarbon feedstream with a catalyst comprising a hydrogenation metal in the presence of sulfur wherein the sulfur is introduced after catalyst start-up and thereafter continuously introduced for a period of more than 1 day at a concentration of no more than 56 ppm by weight of the aromatic hydrocarbon feedstream.

The process is particularly beneficial during periods of sub-optimal highly reactive non-start-up reaction conditions. Thus, the process may be performed with a weight hourly space velocity (WHSV) of aromatic hydrocarbon feedstream less than 8 hr⁻¹, or alternatively less than 80% of an optimal aromatic hydrocarbon WHSV for isomerization of aromatic hydrocarbons in the reactor. The process may also comprise a reactor in which the contacting occurs, the reactor operating with a hydrogen partial pressure greater than 125 psia, or alternatively greater than 110% of an optimal hydrogen partial pressure for isomerization of aromatic hydrocarbons in the reactor.

Alternatively, the inventive continuous process for the isomerization of aromatic hydrocarbons may comprise contacting an aromatic C8 feedstream comprising ethylbenzene with hydrogen and a catalyst comprising a hydrogenation metal; continuously introducing sulfur at a concentration of no more than 200 ppm by weight of the aromatic hydrocarbon feedstream for a period of time after catalyst start-up; producing a product stream having a higher proportion of paraxylene when compared with the aromatic hydrocarbon feedstream with an average ethylbenzene conversion of at least 70% and a ring loss of no more than 2 mol % while sulfur is present.

It has been discovered that the inventive process may be performed to decrease ring loss substantially without significant or permanent damage to the overall activity of catalyst. Thus, the inventive process may further embody an average ethylbenzene conversion for the production of product stream in the presence of sulfur which is at most 3% less than the average ethylbenzene conversion for the production of product stream before sulfur is introduced, while the ring loss for the production of product stream in the presence of sulfur is at least 0.1 mol % less the average ring loss for the production of product stream before sulfur is introduced. Alternatively, the inventive process may comprise discontinuing the introduction of sulfur; wherein the average ethylbenzene conversion for the production of product stream after the introduction of sulfur is discontinued is at most 1.5% less than the average ethylbenzene conversion for the production of product stream before sulfur is introduced, and the ring loss for the production of product stream after the introduction of sulfur is discontinued is at least 0.3 mol % less than the average ring loss for the production of product stream before sulfur is introduced.

These and other objects, features, and advantages will become apparent as reference is made to the following detailed description, preferred embodiments, examples, and appended claims.

### BRIEF DESCRIPTION OF THE DRAWINGS

FIGS. 1-2 are experimental results illustrating some advantages of the present invention.

### DETAILED DESCRIPTION

Described herein are methods and systems for the isomerization of xylenes with post-oil-in sulfidation. Various specific aspects of the invention will now be described, including definitions that are adopted herein for purposes of understanding the claimed invention. While the following detailed description illustrates specific aspects, those skilled in the art will appreciate that the invention can be practiced in other ways. For purposes of determining infringement, the scope of the invention will refer to any one or more of the appended claims, including their equivalents, and elements or limitations that are equivalent to those that are recited. Any reference to the “invention” may refer to one or more, but not necessarily all, of the inventions defined by the claims.

A process is provided for the isomerization of aromatic hydrocarbons which comprises contacting an aromatic hydrocarbon feedstream with a catalyst including a hydrogenation metal in the presence of sulfur wherein the sulfur is introduced after catalyst start-up and thereafter continuously introduced for a period of more than 1 day at a concentration of no more than 56 ppm by weight of the aromatic hydrocarbon feedstream.

The aromatic hydrocarbon feedstream preferably includes an aromatic C8 mixture containing ethylbenzene and at least one xylene isomer and typically all three of the xylene isomers. The feedstream may be derived from catalytic reforming of petroleum naphtha. The feedstream may be para-xylene depleted, meaning that the concentration of para-xylene in the feedstream, relative to its C8 isomers, is lower than the thermodynamic equilibrium concentration of para-xylene in a mixture of C8 isomers, for example less than 23 mol % paraxylene based on the total C8 aromatic hydrocarbons content of the feedstream. The paraxylene depleted feedstream may originate from a paraxylene separation unit, e.g., Parex™ Unit or Eluxyl™ Unit or crystallization unit. The feedstream may have an ethylbenzene content in the approximate range of 1 to 60 wt %, an ortho-xylene content in the approximate range of 0 to 35 wt %, a meta-xylene content in the approximate range of 20 to 95 wt % and a para-xylene range of 0 to 15 wt %. Additionally, the aromatic hydrocarbon feedstream may contain non-aromatic hydrocarbons, e.g., naphthenes and paraffins, such as in an amount up to 30 wt %.

The catalyst includes a hydrogenation component, such as provided by one or more metals selected from Group 7 (e.g., rhenium) or Groups 8-10 (e.g., platinum; formerly “Group VIII”), using the modern designations of the groups in the Periodic Table. The hydrogenation metal is preferably rhenium (Re) or platinum (Pt). The catalyst may also include a molecular sieve, and a support such as alumina or clay. The catalyst may be molded by known methods known in the art per se, such as extrusion molding, compression molding, and rolling moldings.

Alternatively, the catalyst system for the invention may comprise at least two catalysts, where two catalysts each include a hydrogenation metal, molecular sieve, and support as described above. In such a system, the first catalyst has the primary function of selectively de-ethylating the ethylbenzene in the feedstream to benzene, and the second catalyst primarily isomerizes xylenes in the feed. Catalyst systems and the respective components are per se known in the art and can be selected by one of ordinary skill in the art in possession of the present disclosure. Particularly preferred systems are disclosed, for instance, in U.S. Pat. Nos. 5,516,956; 6,028,238; and 8,835,705, mindful of the teachings of the present disclosure.

In general, the process of the invention is carried out in a fixed bed reactor containing the catalyst system described above. The catalyst may be pre-sulfided, either before installation in the reactor, as is known in the art, or in the reactor prior to introduction of hydrocarbon feedstream. The first and second components of the catalyst system may be in sequential beds in a single reactor. That is, the component of the catalyst system used in the process which is primarily effective for ethylbenzene conversion may form a first bed, while the other component of the catalyst system, which is primarily effective for xylene isomerization, may form a second bed downstream of the first bed. As an alternative, the first and second beds may be disposed in separate reactors which, if desired, may be operated at different process conditions. Additional catalyst beds may be provided prior or after the first and second catalyst components of the invention.

When the catalyst system is contacted with the feedstream, the conditions used in the process of the invention are not narrowly defined, but generally will include a temperature of from about 400 to about 1,000° F. (about 204° C. to about 537° C.), a pressure of from about 0 to about 1,000 psig (6.895 MPa-g), a WHSV of between about 0.1 and about 200 hr⁻¹, and a hydrogen, H₂, to hydrocarbon, HC, molar ratio of between about 0 and about 10. Preferably, the conditions include a temperature of from about 650 to about 878° F. (about 340-470° C.), a pressure of from about 50 and about 400 psig (about 0.34 to 2.76 MPa-g), a WHSV of between about 3 and about 50 hr⁻¹ and a H₂ to HC molar ratio of between about 0.5 and about 5.

Sulfur is introduced into the reactor after catalyst start-up (i.e., oil-in) and is thereafter continuously introduced for a period of time concurrently with the aromatic hydrocarbon feed. The sulfur treatment, i.e., co-sulfiding may comprise flowing hydrogen which contains H₂S gas at elevated temperature such as from above room temperature to about 500° C., preferably 100° C. to 450° C. Alternatively, liquid DMDS (dimethyl disulfide) may be injected into the reactor or co-fed with the aromatic hydrocarbon feed. DMDS decomposes to H₂S and methane once entering the reactor. Sulfur is preferably introduced in relatively small concentrations. For example, given as ppm by weight of H₂S in relation to the aromatic hydrocarbon feed, sulfur may be introduced at a concentration from over 0 wppm to under 100 wppm. Preferably sulfur is introduced at a concentration from 1 wppm to 56 wppm, or alternatively at most 20 wppm, or at most 10 wppm, or at most 5 wppm. Introduction of sulfur may be controlled so that concentration is constant over the course of the sulfiding treatment. Alternatively, sulfur concentration may be variably controlled over the course of treatment, either increasing or decreasing, gradually or in step-wise changes. For example, sulfur may be introduced initially at a concentration of 10 wppm for a period of time and then introduced at a concentration of 5 wppm for a subsequent period of time before non-sulfided operations are recommenced.

Sulfur is introduced after catalyst start-up during otherwise non-sulfided operation. This however does not preclude that the catalyst was previously subject to sulfiding/passivating, either before, during, or for a short time after catalyst start-up, but in such situations, sulfiding is discontinued for a discrete period of reactor operation before introducing sulfur according to the invention.

For various reasons, it may be necessary to run the isomerization reaction at sub-optimal highly reactive conditions after reactor start-up. For instance, upstream or downstream process constraints, equipment maintenance and/or failure, or reactant supply/accessibility may all necessitate operating xylene isomerization at relatively low aromatic hydrocarbon feed rate or relatively high hydrogen partial pressure. Sulfur may be advantageously introduced according to the invention during these periods of sub-optimal highly reactive non-start-up reaction conditions. Thus, the inventive process may have a WHSV of aromatic hydrocarbon feedstream less than 8 hr⁻¹, or more preferably less than 6 hr⁻¹. Also, the reactor may operate with a hydrogen partial pressure greater than 125 psia, or more preferably greater than 140 psia. Alternatively, the inventive process may have a WHSV of aromatic hydrocarbon feedstream less than 80%, or more preferably less than 60% of an optimal aromatic hydrocarbon WHSV for isomerization of aromatic hydrocarbons in the utilized reactor. Alternatively the reactor may operate with a hydrogen partial pressure greater than 110%, or more preferably greater than 120% of an optimal hydrogen partial pressure for isomerization of aromatic hydrocarbons in the utilized reactor. Optimal isomerization reaction conditions such as “optimal aromatic hydrocarbon WHSV” and “optimal hydrogen partial pressure” as used above vary between each individual reactor depending on type, size, catalyst, hydrocarbon feed, etc., but it is generally within the skill of the art to determine such optimal isomerization reaction conditions for a given reaction setup through preliminary calculation and limited, reasonable experimentation. By way of example, for a given isomerization reactor setup with an optimal aromatic hydrocarbon WHSV of 10 hr⁻¹ and optimal hydrogen partial pressure of 115 psia, the invention may be advantageously performed at a WHSV less than 8 hr⁻¹¹ (80% of optimal value), or more preferably less than 6 hr⁻¹ (60% of optimal value above), and alternatively or additionally performed at a hydrogen partial pressure of 125 psia (approximately 110% above optimal hydrogen partial pressure), or more preferably greater than 140 psia (approximately 20% above optimal hydrogen partial pressure). The sub-optimal highly reactive process conditions may be consistent or intermittent over the course of the catalyst cycle, or alternatively temporary such as in the case when the prevailing cause of the sub-optimal process conditions resolved or changed.

Introduction of sulfur according to the invention may advantageously be performed for long periods of time, for instance more than one day, preferably between one day and 365 days.

The activity of the isomerization catalyst may be indicated by a variety of measurements, including ethylbenzene conversion, ring loss, or metal efficiency. An exemplary measure of catalyst activity particularly suitable with regard to the invention is ethylbenzene conversion. Higher ethylbenzene conversion is desirable in order to minimize ethylbenzene concentration in the xylene loop system. Ring loss indicates the amount of aromatic compounds that are saturated during the isomerization process. It has been found that during sub-optimal periods of non-start-up high reactivity, co-sulfiding according to the present invention can result in substantially decreased ring loss without substantially decreasing ethylbenzene conversion. Thus, in an aspect of the invention, the process may comprise producing a product stream with an average ethylbenzene conversion of at least 70 wt %, or more preferably at least 80 wt %, or even more preferably at least 85 wt %. In another aspect of the invention, the process may comprise producing a product stream with a ring loss of no more than 2 mol %, or more preferably no more than 1.8 mol %, or even more preferably no more than 1.3 mol % while sulfur is present. These aspects may be combined such that the process may comprise producing a product stream with an average ethylbenzene conversion of at least 70 wt % and a ring loss of no more than 2 mol %. More preferably the process comprises producing a product stream with an average ethylbenzene conversion of at least 85 wt % and a ring loss of no more than 1.3 mol %.

It has been discovered that the inventive co-sulfiding process may decrease ring loss in the isomerization process without significant long term effect on catalyst activity as shown by only a slight decrease in ethylbenzene conversion in the isomerization process according to the invention compared to isomerization process without sulfur, assuming reactor temperature is equal. Thus, the inventive process may be characterized by an average ethylbenzene conversion for the production of product stream in the presence of sulfur which is at most 3%, or preferably at most 1%, less than the average ethylbenzene conversion for the production of product stream before sulfur is introduced, while the ring loss for the production of product stream in the presence of sulfur is at least 0.1 mol %, or preferably at least 0.2 mol %, or more preferably at least 0.3 mol %, below the average ring loss for the production of product stream before sulfur is introduced.

Additionally, it has been surprisingly discovered that ring loss is beneficially lowered without significant long term effect on activity even after introduction of sulfur has been discontinued. Thus the process may further be characterized by having an average ethylbenzene conversion for the production of product stream after the introduction of sulfur is discontinued of at most 1.5%, preferably at most 1%, and more preferably at most 0.5% less than the average ethylbenzene conversion for the production of product stream before sulfur is introduced, while having a ring loss for the production of product stream after the introduction of sulfur is discontinued of at least 0.3 mol %, preferably at least 0.4 mol % less than the average ring loss for the production of product stream before sulfur is introduced.

After the conversion process, the isomerization product may comprise a mixture of C8 aromatic hydrocarbons having a reduced ethylbenzene content and increased paraxylene content relative to the feedstream. The isomerization product may then be treated to isolate paraxylene and/or other desirable xylene(s). Thus, for example, the isomerate product can be fed to a variety of paraxylene recovery units, such as a crystallizer, a membrane separation unit, or a selective adsorption unit (e.g., Parex™ unit), and thus, the paraxylene may be isolated and recovered, leaving a paraxylene depleted C8 aromatic hydrocarbon by-product or residual isomerate. The residual isomerate can be stripped of products lighter than C8 aromatic hydrocarbons. Products heavier than C8 aromatic hydrocarbons in the residual isomerate can be further processed or may be fractionated out. C8 aromatic hydrocarbon fractions from which para-xylene has been removed can be recycled to the process.

The following examples are intended to be representative and not limiting of the present invention.

### EXAMPLE

An isomerization process was conducted at constant residence time and hydrogen partial pressure (after catalyst de-edging) while introducing sulfur in a range of concentrations. This was repeated at two different average reactor temperatures. Specifically, the reactor was operated during defined, sequential periods of time ranging from ˜1.5 days to ˜8 days (Periods I-XIV) according to set process conditions. These reaction conditions simulate sub-optimal periods of non-start-up high activity. During each Period, ethylbenzene conversion and ring loss were determined For all Periods I-XIV, the aromatic hydrocarbon feedstream WHSV was set at 10 hr⁻¹. The process conditions, ethylbenzene conversion and ring loss for each Period appear below. Period I is a start-up phase in which the catalyst was de-edged. Period II constituted “base conditions #1” while Period IX constituted “base conditions #2”.

The experiments were carried out in a pilot plant unit equipped with a reactor, a feed system, an effluent collection and analytic system. The reactor had a catalyst basket capable of holding a catalyst volume of up to 100 mL. The mixed xylenes were pumped into a vaporizer and mixed with H₂ stream and/or other dilution gas, gas tracers etc., and then fed into the reactor in a downflow pattern. The reactor effluent was condensed and collected in a liquid drum while the light gas was directed to the flare line. A thermal well was equipped in the reactor to allow a traveling thermocouple to record the catalyst temperature along the bed axis, so that an average reactor temperature (ART) could be obtained. A gas chromatography sampling system was equipped to take on-line samples for product analysis on a frequent basis when sulfur compounds were not present in the effluent. However, because the on-line gas chromatography sampling system was not compatible with sulfur-containing streams, when H₂S gas was co-fed into the reactor system, a time-average liquid sample was collected in the product drum and a light gas sample was also collected during the same time period. Both the time-average liquid sample and light gas sample were analyzed with an off-line gas chromatography system and a material balance was performed to determine ethylbenzene conversion, product yields and ring loss, etc.

TABLE 1

| | | | | | Ethyl- | |
|---|---|---|---|---|---|---|
| | Avg. | Avg. | H₂ Partial | Sulfur | benzene | Ring |
| | Temp. | Pressure | Pressure | Conc. | Conversion | Loss |
| Period | (° F.) | (psig) | (psia) | (wppm) | (wt %) | (mol %) |
| I | 789 | 187 | 95 | 0 | — | 0.24 |
| II | 772 | 369 | 170 | 0 | 85.9 | 1.24 |
| | (BC1) | (BC1) | (BC1) | | | |
| III | BC1 | BC1 | BC1 | 1 | 85.6 | 1.27 |
| IV | BC1 | BC1 | BC1 | 5 | 85.2 | 1.11 |
| V | BC1 | BC1 | BC1 | 10 | 85.2 | 1.01 |
| VI | BC1 | BC1 | BC1 | 20 | 85.0 | 1.01 |
| VII | BC1 | BC1 | BC1 | 56 | 84.4 | 0.75 |
| VIII | BC1 | BC1 | BC1 | 0 | 85.4 | 0.88 |
| IX | 731 | 369 | 170 | 0 | 77.2 | 2.02 |
| | (BC2) | (BC2) | (BC2) | | | |
| X | BC2 | BC2 | BC2 | 10 | 76.7 | 1.79 |
| XI | BC2 | BC2 | BC2 | 20 | 76.7 | 1.69 |
| XII | BC2 | BC2 | BC2 | 56 | 74.4 | 1.49 |
| XIII | BC2 | BC2 | BC2 | 0 | 75.0 | 1.83 |
| XIV | BC1 | BC1 | BC1 | 0 | 84.7 | 0.93 |

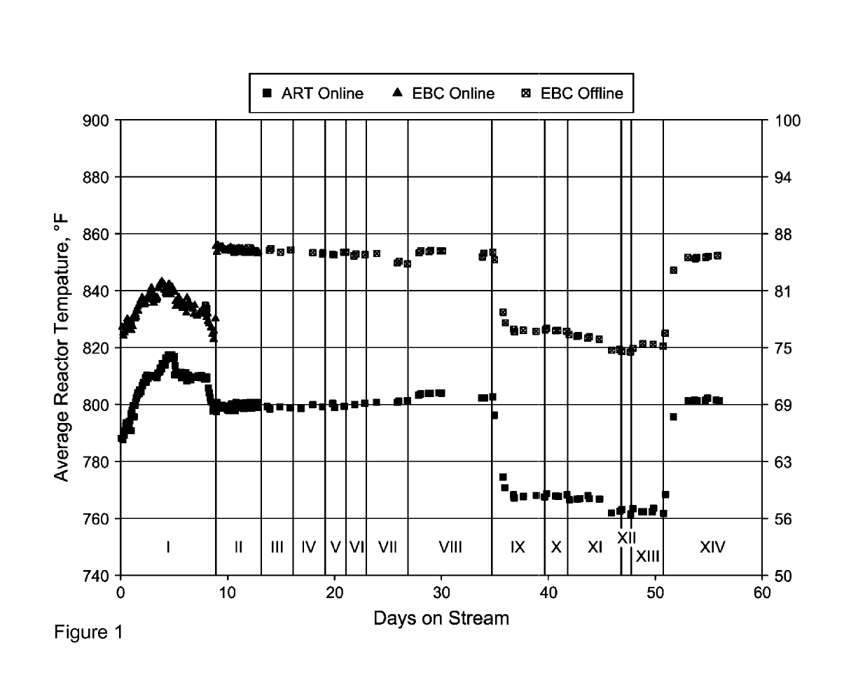

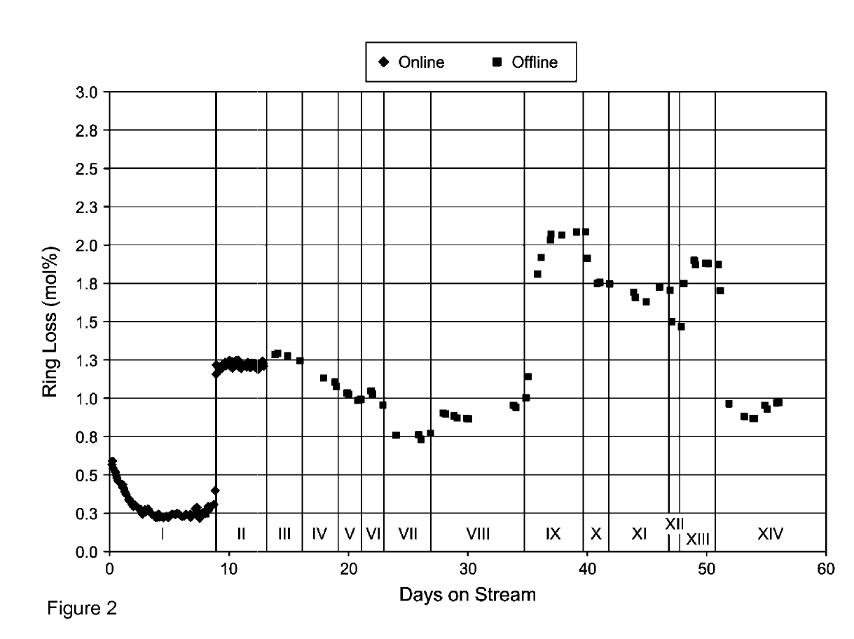

FIG. 1 shows ethylbenzene conversion and average reactor inlet temperature as a function of time of reaction in terms of days on stream with demarcation of Periods I-XIV. FIG. 2 shows ring loss measured during the same period of time, also showing demarcation of Periods I-XIV.

As can be seen in Table 1 and FIGS. 1 and 2, by Period VIII when H2S has been removed from the feed, sulfur has been co-injected for an extended period of time but when sulfur co-feeding is interrupted, ethylbenzene conversion returns close to its initial level at Period II (base conditions #1), but ring loss is substantially less than that at Period II. A drop in ethylbenzene conversion is noted at Period IX when base conditions #2 are established. This drop is directly related to the decrease in reactor inlet temperature under base conditions #2. At Period XIII, H₂S is removed from the feed again. As can be seen, sulfur has been co-injected for an extended period of time but when sulfur co-feeding is interrupted, ethylbenzene conversion increases. Ring loss also increases, but remains substantially lower than that of Period IX at base conditions #2. At Period XIV, sulfur has been co-injected at two different temperatures and for an extended period each time, but after conditions return to initial base conditions, ethylbenzene conversion returns to close to its initial level (84.7% vs. 85.9% initially) while ring loss is substantially lower than the initial level.

Thus, even with ˜40% of the run operated with some level of co-sulfiding, catalyst activity is surprisingly not significantly affected vs. initial activity. Concurrently, undesirable ring loss was significantly reduced during co-sulfiding periods, illustrating that the inventive method efficiently reduces ring loss during periods of sub-optimal high activity conditions. Moreover, long co-sulfiding periods according to the invention show a surprising advantageous permanent impact on ring loss, which is lower than initial levels even after sulfur co-feeding is suppressed.

The present invention may be utilized in any number of diverse integrated systems including integration with other aromatics conversion processes such as transalkylation, disproportionation, alkylation, and mixtures thereof, with other petrochemical, refining, and chemical operations.

Trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated. While the illustrative embodiments of the invention have been described with particularity, it will be understood that various other modifications will be apparent to and can be readily made by those skilled in the art without departing from the spirit and scope of the invention.

What is claimed is:

1. A continuous process for the isomerization of aromatic hydrocarbons comprising contacting an aromatic hydrocarbon feedstream with a catalyst comprising a hydrogenation metal in the presence of sulfur;
wherein the sulfur is introduced after catalyst start-up and thereafter continuously introduced for a period of more than 1 day at a concentration of no more than 200 ppm by weight of the aromatic hydrocarbon feedstream.

2. A process according to claim 1 wherein the sulfur is introduced for a period between 1 day and 50 days.

3. A process according to claim 1 wherein the sulfur is introduced more than 1 day after catalyst start-up.

4. A process according to claim 1 wherein the sulfur is continuously introduced until the end of a catalyst cycle.

5. A process according to claim 1 wherein the weight hourly space velocity of the aromatic hydrocarbon feedstream is less than 6 hr⁻¹.

6. A process according to claim 1 wherein the weight hourly space velocity of the aromatic hydrocarbon feedstream is less than 60% of an optimal aromatic hydrocarbon feed rate for isomerization of aromatic hydrocarbons in the reactor.

7. A process according to claim 1 wherein the contacting occurs in a reactor operating with a hydrogen partial pressure greater than 140 psia.

8. A process according to claim 1 wherein the contacting occurs in a reactor operating with a hydrogen partial pressure greater than 120% of an optimal hydrogen partial pressure for isomerization of aromatic hydrocarbons in the reactor.

9. A process according to claim 1 wherein the sulfur is continuously introduced at a concentration of no more than 10 ppm by weight of the aromatic hydrocarbon feedstream.

10. A process according to claim 1 wherein the sulfur is continuously introduced at a concentration of no more than 5 ppm by weight of the aromatic hydrocarbon feedstream.

11. A process according to claim 1 wherein the sulfur is present in the form of hydrogen sulfide.

12. A process according to claim 1 wherein the sulfur is introduced in the form of dimethyl disulfide, which thereafter decomposes into hydrogen sulfide.

13. A process according to claim 1 wherein the sulfur is introduced in a mixture of hydrogen sulfide and hydrogen gas.

14. A process according to claim 1 wherein the aromatic hydrocarbon feed comprises ethylbenzene and a paraxylene-depleted feedstream of aromatic hydrocarbons, the process further comprising:
producing a product having a higher proportion of paraxylene when compared with the aromatic hydrocarbon feedstream.

15. A continuous process for the isomerization of aromatic hydrocarbons comprising:
contacting an aromatic C8 feedstream comprising ethylbenzene with hydrogen and a catalyst comprising a hydrogenation metal;
continuously introducing sulfur at a concentration of no more than 200 ppm by weight of the aromatic hydrocarbon feedstream for a period of time after catalyst start-up; and
producing a product stream having a higher proportion of paraxylene when compared with the aromatic hydrocarbon feedstream with an average ethylbenzene conversion of at least 70% and a ring loss of no more than 2 mol % while sulfur is present.

16. A process according to claim 15 wherein the product stream is produced with an average ethylbenzene conversion of at least 85% and a ring loss of no more than 1.2 mol %.

17. A process according to claim 15 wherein the average ethylbenzene conversion for the production of product stream in the presence of sulfur is at most 3% less than the average ethylbenzene conversion for the production of product stream before sulfur is introduced, and the ring loss for the production of product stream in the presence of sulfur is at least 0.1 mol % less the average ethylbenzene conversion for the production of product stream before sulfur is introduced.

18. A process according to claim 15 wherein the average ethylbenzene conversion for the production of product stream in the presence of sulfur is at most 1% less than the average ethylbenzene conversion for the production of product stream before sulfur is introduced, and the ring loss for the production of product stream in the presence of sulfur is at least 0.2 mol % less than the average ethylbenzene conversion for the production of product stream before sulfur is introduced.

19. A process according to claim 15 further comprising:
discontinuing the introduction of sulfur;
wherein the average ethylbenzene conversion for the production of product stream after the introduction of sulfur is discontinued is at most 1.5% less than the average ethylbenzene conversion for the production of product stream before sulfur is introduced, and the ring loss for the production of product stream after the introduction of sulfur is discontinued is at least 0.3 mol % less than the average ethylbenzene conversion for the production of product stream before sulfur is introduced.
